(19) United States
(12) Patent Application Publication
(10) Pub. No.: US 2011/0207144 A1
(43) Pub. Date: Aug. 25, 2011

(54) IN VITRO SCREENING ASSAYS

(76) Inventors: Derek Marshall, San Francisco, CA (US); Victoria Smith, Burlingame, CA (US)

(21) Appl. No.: 12/860,632

(22) Filed: Aug. 20, 2010

Related U.S. Application Data
(60) Provisional application No. 61/235,796, filed on Aug. 21, 2009.

(51) Int. Cl. C12Q 1/68; G01N 33/53; C12Q 1/02

(52) U.S. Cl. 435/6.13; 435/7.1; 435/29

(57) ABSTRACT

Disclosed herein are methods for identifying inhibitors of the catalytic activity of LOXL2 using in vitro assays. In certain embodiments, inhibitors identified by these in vitro assays are effective in inhibiting tumor desmoplasia and fibrosis.

### CROSS REFERENCE TO RELATED APPLICATIONS

This application claims priority benefit to U.S. provisional application Ser. No. 61/235,796, filed Aug. 21, 2009, which application is incorporated herein by reference in its entirety for all purposes.

This application is related to co-owned U.S. provisional application Ser. No. 61/235,852, filed Aug. 21, 2009 and to co-owned United States application entitled “Therapeutic Methods and Compositions,” Attorney Docket No. ARBS-011, Client Reference No. A11-US1, filed even date herewith; the disclosures of which are incorporated by reference in their entireties for all purposes.

### STATEMENT REGARDING FEDERAL SUPPORT

Not applicable.

### FIELD

The present disclosure is in the field of screening assay useful for the identification of molecules for the treatment of various disorders in which connective tissue plays a role, including, for example, cancer and fibrosis.

### BACKGROUND

A number of in vitro assays have been used to study aspects of tumor development and metastasis. There are also a number of in vitro model systems for the study of chemically-induced fibrosis. The extracellular matrix enzyme lysyl oxidase-like protein-2 (LOXL2) has been shown to play a role in both processes. See, for example, WO 2004/047720 (Jun. 10, 2004); US 2006/0127402 (Jun. 15, 2006); US 2009/0053224 (Feb. 26, 2009); US 2009/0104201 (Apr. 23, 2009); Kirschmann et al. (2002) Cancer Research 62:4478-4483. Thus, the lysyl oxidase-like-2 enzyme represents an important therapeutic target. Hence, methods to screen for inhibitors of LOXL2 would be desirable.

### SUMMARY

Disclosed herein are methods and compositions for the use of various in vitro assays to identify inhibitors of LOXL2 that are effective in blocking the desmoplastic and fibrotic activities of this enzyme. Accordingly, the disclosure provides, inter alia:

1. A method for identifying an inhibitor of LOXL2 activity, the method comprising:
(a) providing a co-culture of endothelial cells growing in the presence of stromal cells;
(b) adding a test molecule to the co-culture; and
(c) assaying the co-culture for angiogenesis or vasculogenesis;

wherein a test molecule that reduces the degree of angiogenesis or vasculogenesis in the co-culture, compared to a co-culture in the absence of the test molecule, is identified as an inhibitor of LOXL2 activity.

2. The method of embodiment 1, wherein the endothelial cells are human endothelial cells.

3. The method of embodiment 2, wherein the endothelial cells are human umbilical vein endothelial cells (HUVEC).

4. The method of embodiment 1, wherein the stromal cells are human cells.

5. The method of embodiment 1, wherein the stromal cells are fibroblasts.

6. The method of embodiment 1, wherein the test molecule is a polypeptide.

7. The method of embodiment 6, wherein the polypeptide is an antibody.

8. The method of embodiment 7, wherein the antibody is an anti-LOXL2 antibody.

9. The method of embodiment 1, wherein the test molecule is a nucleic acid.

10. The method of embodiment 9, wherein the nucleic acid is a siRNA.

11. The method of embodiment 1, wherein the test molecule is a small organic molecule with a molecular weight less than 1,000 D.

12. The method of embodiment 1, wherein reduction in the degree of angiogenesis or vasculogenesis is indicated by a reduction in vessel number or density.

13. The method of embodiment 1, wherein reduction in the degree of angiogenesis or vasculogenesis is indicated by a reduction in vessel length.

14. The method of embodiment 1, wherein reduction in the degree of angiogenesis or vasculogenesis is indicated by a reduction in the degree of vessel branching.

15. The method of embodiment 1, wherein reduction in the degree of angiogenesis or vasculogenesis is indicated by a reduction in the levels of CD31.

### BRIEF DESCRIPTION OF THE DRAWINGS

FIG. 1 is a representative image of HUVEC treated with 50 ug/ml AB0023 and stained for CD31 expression.

FIG. 2 is a representative image of HUVEC treated with 20 uM suramin and stained for CD31 expression.

FIG. 3 is a representative image of untreated HUVEC stained for CD31 expression.

FIG. 4 is a representative image of HUVEC treated with 2 ng/ml vascular endothelial growth factor (VEGF) and stained for CD31 expression.

### DETAILED DESCRIPTION

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N.J., 2000; and the series “Methods in Enzymology,” Academic Press, San Diego, Calif.

Angiogenesis Assays

In certain embodiments, screens for LOXL2 inhibitors rely on the ability of LOXL2 to promote angiogenesis. See, for example, WO 02/11667. Accordingly, in certain embodiments, a test substance is identified as a LOXL2 inhibitor by virtue of its ability to inhibit angiogenesis in an in vitro angiogenesis assay. Various assays of this type are available (see, for example, Ribatti & Vacca (1999) Intl. J. Biol. Markers 14:207-213); and exemplary assays are now described.

Chick Chorioallantoic Membrane

The extraembryonic chorioallantoic membrane of the chick is formed by the fusion of the chorion and the allantois. It is in direct contact with the shell and contains a thick capillary network. The assay can be conducted in ovo, in which case a window is cut into the shell to allow viewing of the membrane. Alternatively, removal of the embryo and its associated membranes to a culture vessel allows an in vitro assay to be performed. See, for example, Auerbach et al. (1974) Devel. Biol. 41:391-394; the disclosure of which is incorporated by reference in its entirety. In either case, a test substance is applied to the membrane and its effect on angiogenesis is observed and/or measured. In certain embodiments, a test substance is administered to the membrane in a biologically inert polymer that allows controlled and/or sustained release of the test substance. Examples of such polymers include, but are not limited to, ethylene vinyl acetate copolymers (e.g., Elvax 40) and poly-2-hydroxyethyl methacrylate polymers (e.g., hydron). Collagen gels or gelatin sponges, impregnated with test substance, can also be used. See, for example, Nguyen et al. (1994) Microvascular Res. 47:31-40; Ribatti et al. (1997) J. Vascular Res. 34:455-463; the disclosures of which are incorporated by reference in their entireties. Modifications and adaptations of this assay have been described. See, for example, Parsons-Wingerter et al. (2000) Arteriosclerosis Thrombosis Vasc. Biol. 20:1250-1256; Gonzalez-Iriate et al. (2003) Angiogenesis 6:251-254; Miller et al. (2004) J. Translational Med. 2:4; the disclosures of which are incorporated by reference in their entireties.

See also Ribatti et al. (1996) Intl. J. Devel. Biol. 40:1189-1197; Ribatti et al. (2000) Curr. Pharmacol. Biotechnol. 1:72-73; Richardson & Singh (2003) Curr. Drug Targets Cardiovasc. Hematol. Disorders 3:155-185; and Ribatti (2004) Leukemia 18:1350-1351, the disclosures of which are incorporated by reference for the purpose of describing the chick chorioallantoic membrane assay.

Cornea Pocket Assays

In this assay, slow-release pellets containing a test substance are implanted into the corneal stroma of the nonvascularized rabbit eye. Any vessel formation detected is due to newly formed blood vessels. See, for example, Hartwell (1998) Microcirculation 5:173-178; Presta et al. (1999) Cancer Res. 59:2417-2424; Ribatti & Vacca (1999) supra; Morbidelli & Ziche (2004) Cancer Treatment Res. 117:147-151; the disclosures of which are incorporated by reference in their entireties for the purpose of describing these cornea-based assays. Similar assays, using the rat eye, have also been described.

Human Co-Culture Angiogenesis Assays

This assay utilizes primary endothelial cells and primary fibroblasts. Development of vascular structures in the presence of one or more test substances is determined. See, for example, Beilmann et al. (2004) Cytokine 26:178-185; the disclosure of which is incorporated by reference in its entirety for the purpose of describing human co-culture assays.

Aortic Ring Assays

In this system, an aorta (e.g., from a rat) is cut into small disks and cultured, e.g., in a fibrin matrix. Migration of endothelial cells and/or smooth muscle cells from the cut edge produces radial outgrowth of microvessels. See, for example, Nicosia & Ottinetti (1990) Laboratory Investig. 63:115-122; Nissanov et al. (1995) Laboratory Investig. 73:734-739; Blacher et al. (2001) Angiogenesis 4:133-142; Zhu & Nicosia (2002) Angiogenesis 5:81-86; Go & Owen (2003) Meth. Molec. Medicine 85:59-64; the disclosures of which are incorporated by reference in their entireties for the purpose of describing aortic ring assays.

The assay has also been adapted for use with mouse aorta and pig carotid artery. See, for example, Masson V Ve et al. (2002) Biol. Proc. Online 4:24-31; Berger et al. (2004) Microvascular Res. 68:179-187; Stiffey-Wilusz et al. (2001) Angiogenesis 4:3-9; the disclosures of which are incorporated by reference in their entireties for the purpose of describing the aforementioned adaptations.

In another modification of this assay, the miniature ring-supported gel (MRSG) assay, segments of mouse aorta are placed in a three-dimensional extracellular matrix assembly. In one embodiment, small lenticular hydrogels of collagen (e.g., Type I collagen) supported at the edge, e.g., by nylon mesh rings, are used. See, for example, Reed et al. (2007) Microvascular Res. 73:248-252; the disclosure of which is incorporated by reference in its entirety for the purpose of describing the MRSG assay.

Rodent Mesenteric Window

In this assay, angiogenic activity is observed and measured in rodent (e.g., rat) mesentery, an organ that is virtually avascular. See, for example, Norrby (1992) EXS 61:282-286; Jakobsson et al., (1994) Intl. J. Exp. Pathology 75:214-219; the disclosures of which are incorporated by reference in their entireties for the purpose of describing mesenteric window assays.

Placenta Fragment Assays

Fragments of human placental blood vessels, embedded in a fibrin gel, can be used to study angiogenesis. This explant system is particularly useful for the study of human angiogenesis. See, for example, Brown et al. (1996) Laboratory Investigation 75:539-555; the disclosures of which is incorporated by reference in its entirety for the purpose of describing placental fragment assays.

Matrigel Plug Assays

Test substances are introduced into cold Matrigel (BD Biosciences, San Jose, Calif.), an artificial basement membrane which, at 4° C., exists in liquid form. Subcutaneous injection into a host animal (e.g., mouse, rat) results in solidification of the matrix material at body temperature. Penetration of the plug by host cells, and resultant angiogenesis and vasculogenesis can be observed and measured. See, for example, Akhtar et al. (2002); Angiogenesis 5:75-80; Kragh et al. (2003) Intl. J. Oncology 22:305-311; Kragh et al. (2004) Oncology Reports 11:303-307; the disclosures of which are incorporated by reference in their entireties for the purpose of describing Matrigel plug assays.

Micro-Encapsulated Cells

Micro-encapsulated cells, transplanted into experimental animals, can be used to study angiogenic processes. Materials used for encapsulation include, for example, alginate beads, agarose beads, and gelatin-coated microcarriers. See, for example, Nehls & Drenckhahn (1995) Microvascular Res. 50:311-3322; Okada et al. (1995) Japan. J. Cancer Res. 86:1182-1188; the disclosures of which are incorporated by reference in their entireties for the purpose of describing micro-encapsulated cell model systems.

Directed In Vivo Angiogenesis Assays

These assays employ small silicone cylinders, subcutaneously implantated into nude (i.e., athymic) mice, that release substances into surrounding tissues after implantation. See, for example, Guedez et al. (2003) Am. J. Pathol. 162:1431-1439; the disclosure of which is incorporated by reference in its entirety for the purpose of describing directed in vivo angiogenesis assays.

Subcutaneous Air Sac Model System

In this system, subcutaneous injection of air into the back of an experimental animal forms an air sac that induces the appearance of a semi-transparent avascular membrane. Angiogenesis can be observed after implantation of cells or test substances. See, for example, Lichtenberg et al. (1999) Am. J. Pharmacol. Toxicology 84:34-40; the disclosure of which is incorporated by reference in its entirety for the purpose of describing subcutaneous air sac model systems.

Leech Systems

The leech Hirudo medicinalis has been used as a model system for the study of angiogenesis. See, for example, de Eguileor et al. (2004) Current Pharmaceutical Design 10:1979-1998; the disclosure of which is incorporated by reference in its entirety for the purpose of describing leech model systems.

Three-Dimensional Human Tumor Angiogenesis Assay

In this assay, fragments of human tumor tissue are embedded in fibrin gels. Vessels grow into the fibrin gel matrix from the tumor tissue. See, for example, Gulec & Woltering (2004) Ann. Surgical Oncology 11:99-104; the disclosure of which is incorporated by reference in its entirety for the purpose of describing three-dimensional human tumor angiogenesis assays.

Additional information relating to angiogenesis assays can be found in Auerbach et al. (2003) Clinical chemistry 49:32-40 and Norrby (2006) J. Cell. Mol. Med. 10:588-612; the disclosures of which are incorporated by reference in their entireties for the purpose of describing angiogenesis assays.

Markers for Angiogenesis

Morphological, molecular and histochemical markers for angiogenesis are known in the art and include, but are not limited to, vessel formation, CD31 expression (marker for endothelial cells), and expression of von Willebrand Factor (marker for endothelial cells). Assay for any of these, or any other angiogenic markers, can be used to determine degree of vasculogenesis.

Invasion Assays

Invasion/migration assays can also be used to screen for inhibitors of LOXL2, as increased invasiveness and migratory capacity are associated with an epithelial-to-mesenchymal transition (EMT). See, e.g., Bedogni et al. (2004) Cancer Res. 64:2552-2560. Exemplary assays for invasiveness also include, e.g., the Boyden chamber. See, e.g., Chen (2005) Methods Mol. Biol. 294:15-22. In these assays, a test molecule that reduces invasiveness of cells is identified as a LOXL2 inhibitor.

Endothelial Cell-Based Assays

Angiogenesis is a multistep process whereby new blood vessels develop from pre-existing vasculature. Vasculogenesis is a process by which new vessels are formed de novo by migration and condensation of endothelial cells and related stromal cells such as smooth muscle cells and pericytes.

Vessel formation by angiogenesis or vasculogenesis are complex processes with numerous events playing a role, e.g. proteolytic degradation of extracellular matrix, directed migration of endothelial cells, proliferation of endothelial cells, deposition of new extracellular matrix, formation of tubules and anastomosis of the newly formed vessels.

In vitro angiogenesis assays utilizes human endothelial cells (e.g., human umbilical vein endothelial cells, HUVEC) co-cultured with other human cells (e.g., fibroblasts) in a specific matrix. The endothelial cells initially form small islands within the culture matrix and subsequently begin to proliferate and enter a migratory phase during which they move through the matrix to form threadlike tubule structures. After some time (e.g., 9-11 days) they join up and form a network of anastomosing tubules. Therefore this assay recapitulates most aspects of the in vivo processes of vasculogenesis and angiogenesis.

Stromal Cells

In certain of the methods disclosed herein, endothelial cells are co-cultured with stromal cells. Different types of stromal cells, i.e., cells associated with or present in connective tissue, are known in the art and include, for example, fibroblasts, pericytes, smooth muscle cells, mesenchymal stem cells, and mural cells.

Lysyl Oxidase-Type Enzymes

As used herein, the term “lysyl oxidase-type enzyme” refers to a member of a family of proteins that, inter alia, catalyzes oxidative deamination of ε-amino groups of lysine and hydroxylysine residues, resulting in conversion of peptidyl lysine to peptidyl-α-aminoadipic-δ-semialdehyde (allysine) and the release of stoichiometric quantities of ammonia and hydrogen peroxide:

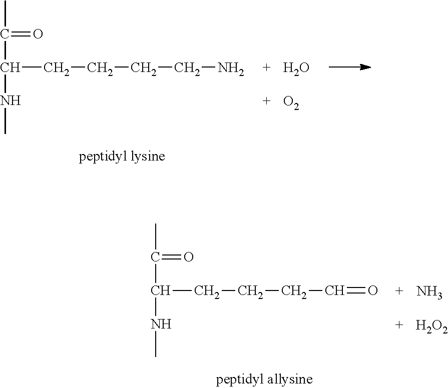

This reaction most often occurs extracellularly, on lysine residues in collagen and elastin. The aldehyde residues of allysine are reactive and can spontaneously condense with other allysine and lysine residues, resulting in crosslinking of collagen molecules to form collagen fibrils.

Lysyl oxidase-type enzymes have been purified from chicken, rat, mouse, bovines and humans. All lysyl oxidase-type enzymes contain a common catalytic domain, approximately 205 amino acids in length, located in the carboxy-terminal portion of the protein and containing the active site of the enzyme. The active site contains a copper-binding site which includes a conserved amino acid sequence containing four histidine residues which coordinate a Cu(II) atom. The active site also contains a lysyltyrosyl quinone (LTQ) cofactor, formed by intramolecular covalent linkage between a lysine and a tyrosine residue (corresponding to lys314 and tyr349 in rat lysyl oxidase, and to lys320 and tyr355 in human lysyl oxidase). The sequence surrounding the tyrosine residue that forms the LTQ cofactor is also conserved among lysyl oxidase-type enzymes. The catalytic domain also contains ten conserved cysteine residues, which participate in the formation of five disulfide bonds. The catalytic domain also includes a fibronectin binding domain. Finally, an amino acid sequence similar to a growth factor and cytokine receptor domain, containing four cysteine residues, is present in the catalytic domain. Despite the presence of these conserved regions, the different lysyl oxidase-type enzymes can be distinguished from one another, both within and outside their catalytic domains, by virtue of regions of divergent nucleotide and amino acid sequence.

The first member of this family of enzymes to be isolated and characterized was lysyl oxidase (EC 1.4.3.13); also known as protein-lysine 6-oxidase, protein-L-lysine:oxygen 6-oxidoreductase (deaminating), or LOX. See, e.g., Harris et al., Biochim. Biophys. Acta 341:332-344 (1974); Rayton et al., J. Biol. Chem. 254:621-626 (1979); Stassen, Biophys. Acta 438:49-60 (1976).

Additional lysyl oxidase-type enzymes were subsequently discovered. These proteins have been dubbed “LOX-like,” or “LOXL.” They all contain the common catalytic domain described above and have similar enzymatic activity. Currently, five different lysyl oxidase-type enzymes are known to exist in both humans and mice: LOX and the four LOX related, or LOX-like proteins LOXL1 (also denoted “lysyl oxidase-like,” “LOXL” or “LOL”), LOXL2 (also denoted “LOR-1”), LOXL3 (also denoted “LOR-2”), and LOXL4. Each of the genes encoding the five different lysyl oxidase-type enzymes resides on a different chromosome. See, for example, Molnar et al., Biochim Biophys Acta. 1647:220-24 (2003); Csiszar, Prog. Nucl. Acid Res. 70:1-32 (2001); WO 01/83702 published on Nov. 8, 2001, and U.S. Pat. No. 6,300,092, all of which are incorporated by reference herein. A LOX-like protein termed LOXC, with some similarity to LOXL4 but with a different expression pattern, has been isolated from a murine EC cell line. Ito et al. (2001) J. Biol. Chem. 276:24023-24029. Two lysyl oxidase-type enzymes, DmLOXL-1 and DmLOXL-2, have been isolated from Drosophila.

Although all lysyl oxidase-type enzymes share a common catalytic domain, they also differ from one another, particularly in their amino-terminal regions. The four LOXL proteins have amino-terminal extensions, compared to LOX. Thus, while human preproLOX (i.e., the primary translation product prior to signal sequence cleavage, see below) contains 417 amino acid residues; LOXL1 contains 574, LOXL2 contains 638, LOXL3 contains 753 and LOXL4 contains 756.

Within their amino-terminal regions, LOXL2, LOXL3 and LOXL4 contain four repeats of the scavenger receptor cysteine-rich (SRCR) domain. These domains are not present in LOX or LOXL1. SRCR domains are found in secreted, transmembrane, or extracellular matrix proteins, and are known to mediate ligand binding in a number of secreted and receptor proteins. Hoheneste et al. (1999) Nat. Struct. Biol. 6:228-232; Sasaki et al. (1998) EMBO J. 17:1606-1613. In addition to its SRCR domains, LOXL3 contains a nuclear localization signal in its amino-terminal region. A proline-rich domain appears to be unique to LOXL1. Molnar et al. (2003) Biochim. Biophys. Acta 1647:220-224. The various lysyl oxidase-type enzymes also differ in their glycosylation patterns.

Tissue distribution also differs among the lysyl oxidase-type enzymes. Human LOX mRNA is highly expressed in the heart, placenta, testis, lung, kidney and uterus, but marginally in the brain and liver. mRNA for human LOXL1 is expressed in the placenta, kidney, muscle, heart, lung, and pancreas and, similar to LOX, is expressed at much lower levels in the brain and liver. Kim et al. (1995) J. Biol. Chem. 270:7176-7182. High levels of LOXL2 mRNA are expressed in the uterus, placenta, and other organs, but as with LOX and LOXL1, low levels are expressed in the brain and liver. Jourdan Le-Saux et al. (1999) J. Biol. Chem. 274:12939:12944. LOXL3 mRNA is highly expressed in the testis, spleen, and prostate, moderately expressed in placenta, and not expressed in the liver, whereas high levels of LOXL4 mRNA are observed in the liver. Huang et al. (2001) Matrix Biol. 20:153-157; Maki and Kivirikko (2001) Biochem. J. 355:381-387; Jourdan Le-Saux et al. (2001) Genomics 74:211-218; Asuncion et al. (2001) Matrix Biol. 20:487-491.

The expression and/or involvement of the different lysyl oxidase-type enzymes in diseases also varies. See, for example, Kagen (1994) Pathol. Res. Pract. 190:910-919; Murawaki et al. (1991) Hepatology 14:1167-1173; Siegel et al. (1978) Proc. Natl. Acad. Sci. USA 75:2945-2949; Jourdan Le-Saux et al. (1994) Biochem. Biophys. Res. Comm. 199:587-592; and Kim et al. (1999) J. Cell Biochem. 72:181-188. Lysyl oxidase-type enzymes have also been implicated in a number of cancers, including head and neck cancer, bladder cancer, colon cancer, esophageal cancer and breast cancer. See, for example, Wu et al. (2007) Cancer Res. 67:4123-4129; Gorough et al. (2007) J. Pathol. 212:74-82; Csiszar (2001) Prog. Nucl. Acid Res. 70:1-32 and Kirschmann et al. (2002) Cancer Res. 62:4478-4483.

Thus, although the lysyl oxidase-type enzymes exhibit some overlap in structure and function, each has distinct structure and functions as well. With respect to structure, for example, certain antibodies raised against the catalytic domain of LOX do not bind to LOXL2. With respect to function, it has been reported that targeted deletion of LOX appears to be lethal at parturition in mice, whereas LOXL1 deficiency causes no severe developmental phenotype. Hornstra et al. (2003) J. Biol. Chem. 278:14387-14393; Bronson et al. (2005) Neurosci. Lett. 390:118-122.

Although the most widely documented activity of lysyl oxidase-type enzymes is the oxidation of specific lysine residues in collagen and elastin outside of the cell, there is evidence that lysyl oxidase-type enzymes also participate in a number of intracellular processes. For example, there are reports that some lysyl oxidase-type enzymes regulate gene expression. Li et al. (1997) Proc. Natl. Acad. Sci. USA 94:12817-12822; Giampuzzi et al. (2000) J. Biol. Chem. 275:36341-36349. In addition, LOX has been reported to oxidize lysine residues in histone H1. Additional extracellular activities of LOX include the induction of chemotaxis of monocytes, fibroblasts and smooth muscle cells. Lazarus et al. (1995) Matrix Biol. 14:727-731; Nelson et al. (1988) Proc. Soc. Exp. Biol. Med. 188:346-352. Expression of LOX itself is induced by a number of growth factors and steroids such as TGF-β, TNF-α and interferon. Csiszar (2001) Prog. Nucl. Acid Res. 70:1-32. Recent studies have attributed other roles to LOX in diverse biological functions such as developmental regulation, tumor suppression, cell motility, and cellular senescence.

Examples of lysyl oxidase (LOX) proteins from various sources include enzymes having an amino acid sequence substantially identical to a polypeptide expressed or translated from one of the following sequences: EMBL/GenBank accessions: M94054; AAA59525.1-mRNA; 545875; AAB23549.1-mRNA; 578694; AAB21243.1-mRNA; AF039291; AAD02130.1-mRNA; BC074820; AAH74820.1-mRNA; BC074872; AAH74872.1-mRNA; M84150; AAA59541.1-Genomic DNA. One embodiment of LOX is human lysyl oxidase (hLOX) preproprotein.

Exemplary disclosures of sequences encoding lysyl oxidase-like enzymes are as follows: LOXL1 is encoded by mRNA deposited at GenBank/EMBL BC015090; AAH15090.1; LOXL2 is encoded by mRNA deposited at GenBank/EMBL U89942; LOXL3 is encoded by mRNA deposited at GenBank/EMBL AF282619; AAK51671.1; and LOXL4 is encoded by mRNA deposited at GenBank/EMBL AF338441; AAK71934.1.

The primary translation product of the LOX protein, known as the prepropeptide, contains a signal sequence extending from amino acids 1-21. This signal sequence is released intracellularly by cleavage between Cys21 and Ala22, in both mouse and human LOX, to generate a 46-48 kDa propeptide form of LOX, also referred to herein as the full-length form. The propeptide is N-glycosylated during passage through the Golgi apparatus to yield a 50 kDa protein, then secreted into the extracellular environment. At this stage, the protein is catalytically inactive. A further cleavage, between Gly168 and Asp169 in mouse LOX, and between Gly174 and Asp175 in human LOX, generates the mature, catalytically active, 30-32 kDA enzyme, releasing a 18 kDa propeptide. This final cleavage event is catalyzed by the metalloendoprotease procollagen C-proteinase, also known as bone morphogenetic protein-1 (BMP-1). Interestingly, this enzyme also functions in the processing of LOX's substrate, collagen. The N-glycosyl units are subsequently removed.

Potential signal peptide cleavage sites have been predicted at the amino termini of LOXL1, LOXL2, LOXL3, and LOXL4. The predicted signal cleavage sites are between Gly25 and Gln26 for LOXL1, between Ala25 and Gln26, for LOXL2, between Gly25 and Ser26 for LOXL3 and between Arg23 and Pro24 for LOXL4.

A BMP-1 cleavage site in the LOXL1 protein has been identified between Ser354 and Asp355. Borel et al. (2001) J. Biol. Chem. 276:48944-48949. Potential BMP-1 cleavage sites in other lysyl oxidase-type enzymes have been predicted, based on the consensus sequence for BMP-1 cleavage in procollagens and pro-LOX being at an Ala/Gly-Asp sequence, often followed by an acidic or charged residue. A predicted BMP-1 cleavage site in LOXL3 is located between Gly447 and Asp448; processing at this site may yield a mature peptide of similar size to mature LOX. A potential cleavage site for BMP-1 was also identified within LOXL4, between residues Ala569 and Asp570. Kim et al. (2003) J. Biol. Chem. 278:52071-52074. LOXL2 may also be proteolytically cleaved analogously to the other members of the LOXL family and secreted. Akiri et al., (2003) Cancer Res. 63:1657-1666.

The sequence of the C-terminal 30 kDa region of the proenzyme in which the active site is located is highly conserved (approximately 95%). A more moderate degree of conservation (approximately 60-70%) is observed in the propeptide domain.

For the purposes of the present disclosure, the term “lysyl oxidase-type enzyme” encompasses all five of the lysine oxidizing enzymes discussed above (LOX, LOXL1, LOXL2, LOXL3 and LOXL4), and also encompasses functional fragments and/or derivatives of LOX, LOXL1, LOXL2, LOXL3 and LOXL4 that substantially retain enzymatic activity; e.g., the ability to catalyze deamination of lysyl residues. Typically, a functional fragment or derivative retains at least 50% of its lysine oxidation activity. In some embodiments, a functional fragment or derivative retains at least 60%, at least 70%, at least 80%, at least 90%, at least 95%, at least 99% or 100% of its lysine oxidation activity.

It is also intended that a functional fragment of a lysyl oxidase-type enzyme can include conservative amino acid substitutions (with respect to the native polypeptide sequence) that do not substantially alter catalytic activity. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz, G. E. and R. H. Schirmer, Principles of Protein Structure, Springer-Verlag, 1979). According to this type of analysis, the following groups of amino acids that can be conservatively substituted for one another can be identified:

(i) amino acids containing a charged group, consisting of Glu, Asp, Lys, Arg and His,

(ii) amino acids containing a positively-charged group, consisting of Lys, Arg and His,

(iii) amino acids containing a negatively-charged group, consisting of Glu and Asp,

(iv) amino acids containing an aromatic group, consisting of Phe, Tyr and Trp,

(v) amino acids containing a nitrogen ring group, consisting of His and Trp,

(vi) amino acids containing a large aliphatic non-polar group, consisting of Val, Leu and Ile,

(vii) amino acids containing a slightly-polar group, consisting of Met and Cys,

(viii) amino acids containing a small-residue group, consisting of Ser, Thr, Asp, Asn, Gly, Ala, Glu, Gln and Pro,

(ix) amino acids containing an aliphatic group consisting of Val, Leu, Ile, Met and Cys, and

(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

For additional information regarding lysyl oxidase-type enzymes, see, e.g., Rucker et al. (1998) Am. J. Clin. Nutr. 67:996 S-1002S and Kagan et al. (2003) J. Cell. Biochem 88:660-672. See also co-owned United States patent application publication Nos. 2009/0053224 (Feb. 26, 2009) and 2009/0104201 (Apr. 23, 2009); the disclosures of which are incorporated by reference herein.

Modulators of the Activity of Lysyl Oxidase-Type Enzymes

Modulators of the activity of lysyl oxidase-type enzymes include both activators (agonists) and inhibitors (antagonists), and can be selected by using a variety of screening assays. The present disclosure presents a number of in vitro assays useful for identifying modulators of the activity of one or more lysyl oxidase-type enzymes.

In additional embodiments, modulators can be identified by determining if a test compound binds to a lysyl oxidase-type enzyme; wherein, if binding has occurred, the compound is a candidate modulator. Optionally, additional tests can be carried out on such a candidate modulator. Alternatively, a candidate compound can be contacted with a lysyl oxidase-type enzyme, and a biological activity of the lysyl oxidase-type enzyme assayed; a compound that alters the biological activity of the lysyl oxidase-type enzyme is a modulator of a lysyl oxidase-type enzyme. Generally, a compound that reduces a biological activity of a lysyl oxidase-type enzyme is an inhibitor of the enzyme.

Other methods of identifying modulators of the activity of lysyl oxidase-type enzymes include incubating a candidate compound in a cell culture containing one or more lysyl oxidase-type enzymes and assaying one or more biological activities or characteristics of the cells. Compounds that alter the biological activity or characteristic of the cells in the culture are potential modulators of the activity of a lysyl oxidase-type enzyme. Biological activities that can be assayed include, for example, lysine oxidation, peroxide production, ammonia production, levels of lysyl oxidase-type enzyme, levels of mRNA encoding a lysyl oxidase-type enzyme, and/or one or more functions specific to a lysyl oxidase-type enzyme. In additional embodiments of the aforementioned assay, in the absence of contact with the candidate compound, the one or more biological activities or cell characteristics are correlated with levels or activity of one or more lysyl oxidase-type enzymes. For example, the biological activity can be a cellular function such as migration, chemotaxis, epithelial-to-mesenchymal transition, or mesenchymal-to-epithelial transition, and the change is detected by comparison with one or more control or reference sample(s). For example, negative control samples can include a culture with decreased levels of a lysyl oxidase-type enzyme to which the candidate compound is added; or a culture with the same amount of lysyl oxidase-type enzyme as the test culture, but without addition of candidate compound. In some embodiments, separate cultures containing different levels of a lysyl oxidase-type enzyme are contacted with a candidate compound. If a change in biological activity is observed, and if the change is greater in the culture having higher levels of lysyl oxidase-type enzyme, the compound is identified as a modulator of the activity of a lysyl oxidase-type enzyme. Determination of whether the compound is an activator or an inhibitor of a lysyl oxidase-type enzyme may be apparent from the phenotype induced by the compound, or may require further assay, such as a test of the effect of the compound on the enzymatic activity of one or more lysyl oxidase-type enzymes.

Methods for obtaining lysysl oxidase-type enzymes, either biochemically or recombinantly, as well as methods for cell culture and enzymatic assay to identify modulators of the activity of lysyl oxidase-type enzymes as described above, are known in the art. See also co-owned United States patent application entitled “Catalytic Domains from Lysyl Oxidase and LOXL2”, Attorney Docket No. ARBS-010, filed even date herewith.

The enzymatic activity of a lysyl oxidase-type enzyme can be assayed by a number of different methods. For example, lysyl oxidase enzymatic activity can be assessed by detecting and/or quantitating production of hydrogen peroxide, ammonium ion, and/or aldehyde, by assaying lysine oxidation and/or collagen crosslinking, or by measuring cellular invasive capacity, cell adhesion, cell growth or metastatic growth. See, for example, Trackman et al. (1981) Anal. Biochem. 113:336-342; Kagan et al. (1982) Meth. Enzymol. 82A:637-649; Palamakumbura et al. (2002) Anal. Biochem. 300:245-251; Albini et al. (1987) Cancer Res. 47:3239-3245; Kamath et al. (2001) Cancer Res. 61:5933-5940; U.S. Pat. No. 4,997,854 and U.S. patent application publication No. 2004/0248871.

Test compounds include, but are not limited to, small organic compounds (e.g., organic molecules having a molecular weight between about 50 and about 2,500 Da), nucleic acids or proteins, for example. The compound or plurality of compounds can be chemically synthesized or microbiologically produced and/or comprised in, for example, samples, e.g., cell extracts from, e.g., plants, animals or microorganisms. Furthermore, the compound(s) can be known in the art but hitherto not known to be capable of modulating the activity of a lysyl oxidase-type enzyme. The reaction mixture for assaying for a modulator of a lysyl oxidase-type enzyme can be a cell-free extract or can comprise a cell culture or tissue culture. A plurality of compounds can be, e.g., added to a reaction mixture, added to a culture medium, injected into a cell or administered to a transgenic animal. The cell or tissue employed in the assay can be, for example, a bacterial cell, a fungal cell, an insect cell, a vertebrate cell, a mammalian cell, a primate cell, a human cell or can comprise or be obtained from a non-human transgenic animal.

Several methods are known to the person skilled in the art for producing and screening large libraries to identify compounds having specific affinity for a target, such as a lysyl oxidase-type enzyme. These methods include the phage-display method in which randomized peptides are displayed from phage and screened by affinity chromatography using an immobilized receptor. See, e.g., WO 91/17271, WO 92/01047, and U.S. Pat. No. 5,223,409. In another approach, combinatorial libraries of polymers immobilized on a solid support (e.g., a “chip”) are synthesized using photolithography. See, e.g., U.S. Pat. No. 5,143,854, WO 90/15070 and WO 92/10092. The immobilized polymers are contacted with a labeled receptor (e.g., a lysyl oxidase-type enzyme) and the support is scanned to determine the location of label, to thereby identify polymers binding to the receptor.

The synthesis and screening of peptide libraries on continuous cellulose membrane supports that can be used for identifying binding ligands of a polypeptide of interest (e.g., a lysyl oxidase-type enzyme) is described, for example, in Kramer (1998) Methods Mol. Biol. 87: 25-39. Ligands identified by such an assay are candidate modulators of the protein of interest, and can be selected for further testing. This method can also be used, for example, for determining the binding sites and the recognition motifs in a protein of interest. See, for example Rudiger (1997) EMBO J. 16:1501-1507 and Weiergraber (1996) FEBS Lett. 379:122-126.

WO 98/25146 describes additional methods for screening libraries of complexes for compounds having a desired property, e.g., the capacity to agonize, bind to, or antagonize a polypeptide or its cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with a lysyl oxidase-type enzyme are, for example, in vitro screening with a phage display system, filter binding assays, and “real time” measuring of interaction using, for example, the BIAcore apparatus (Pharmacia).

All these methods can be used in accordance with the present disclosure to identify activators/agonists and inhibitors/antagonists of lysyl oxidase-type enzymes or related polypeptides.

Another approach to the synthesis of modulators of lysyl oxidase-type enzymes is to use mimetic analogs of peptides. Mimetic peptide analogues can be generated by, for example, substituting stereoisomers, i.e. D-amino acids, for naturally-occurring amino acids; see e.g., Tsukida (1997) J. Med. Chem. 40:3534-3541. Furthermore, pro-mimetic components can be incorporated into a peptide to reestablish conformational properties that may be lost upon removal of part of the original polypeptide. See, e.g., Nachman (1995) Regul. Pept. 57:359-370.

Another method for constructing peptide mimetics is to incorporate achiral o-amino acid residues into a peptide, resulting in the substitution of amide bonds by polymethylene units of an aliphatic chain. Banerjee (1996) Biopolymers 39:769-777. Superactive peptidomimetic analogues of small peptide hormones in other systems have been described. Zhang (1996) Biochem. Biophys. Res. Commun. 224:327-331.

Peptide mimetics of a modulator of a lysyl oxidase-type enzyme can also be identified by the synthesis of peptide mimetic combinatorial libraries through successive amide alkylation, followed by testing of the resulting compounds, e.g., for their binding and immunological properties. Methods for the generation and use of peptidomimetic combinatorial libraries have been described. See, for example, Ostresh, (1996) Methods in Enzymology 267:220-234 and Dorner (1996) Bioorg. Med. Chem. 4:709-715. Furthermore, a three-dimensional and/or crystallographic structure of one or more lysyl oxidase-type enzymes can be used for the design of peptide mimetic inhibitors of the activity of one or more lysyl oxidase-type enzymes. Rose (1996) Biochemistry 35:12933-12944; Rutenber (1996) Bioorg. Med. Chem. 4:1545-1558.

The structure-based design and synthesis of low-molecular-weight synthetic molecules that mimic the activity of native biological polypeptides is further described in, e.g., Dowd (1998) Nature Biotechnol. 16:190-195; Kieber-Emmons (1997) Current Opinion Biotechnol. 8:435-441; Moore (1997) Proc. West Pharmacol. Soc. 40:115-119; Mathews (1997) Proc. West Pharmacol. Soc. 40:121-125; and Mukhija (1998) European J. Biochem. 254:433-438.

It is also well known to the person skilled in the art that it is possible to design, synthesize and evaluate mimetics of small organic compounds that, for example, can act as a substrate or ligand of a lysyl oxidase-type enzyme. For example, it has been described that D-glucose mimetics of hapalosin exhibited similar efficiency as hapalosin in antagonizing multidrug resistance assistance-associated protein in cytotoxicity. Dinh (1998) J. Med. Chem. 41:981-987.

The structure of the lysyl oxidase-type enzymes can be investigated to guide the selection of modulators such as, for example, small molecules, peptides, peptide mimetics and antibodies. Structural properties of a lysyl oxidase-type enzyme can help to identify natural or synthetic molecules that bind to, or function as a ligand, substrate, binding partner or the receptor of, the lysyl oxidase-type enzyme. See, e.g., Engleman (1997) J. Clin. Invest. 99:2284-2292. For example, folding simulations and computer redesign of structural motifs of lysyl oxidase-type enzymes can be performed using appropriate computer programs. Olszewski (1996) Proteins 25:286-299; Hoffman (1995) Comput. Appl. Biosci. 11:675-679. Computer modeling of protein folding can be used for the conformational and energetic analysis of detailed peptide and protein structure. Monge (1995) J. Mol. Biol. 247:995-1012; Renouf (1995) Adv. Exp. Med. Biol. 376:37-45. Appropriate programs can be used for the identification of sites, on lysyl oxidase-type enzymes, that interact with ligands and binding partners, using computer assisted searches for complementary peptide sequences. Fassina (1994) Immunomethods 5:114-120. Additional systems for the design of protein and peptides are described, for example in Berry (1994) Biochem. Soc. Trans. 22:1033-1036; Wodak (1987), Ann. N.Y. Acad. Sci. 501:1-13; and Pabo (1986) Biochemistry 25:5987-5991. The results obtained from the above-described structural analyses can be used for, e.g., the preparation of organic molecules, peptides and peptide mimetics that function as modulators of the activity of one or more lysyl oxidase-type enzymes.

An inhibitor of a lysyl oxidase-type enzyme can be a competitive inhibitor, an uncompetitive inhibitor, a mixed inhibitor or a non-competitive inhibitor. Competitive inhibitors often bear a structural similarity to substrate, usually bind to the active site, and are more effective at lower substrate concentrations. The apparent K_{M} is increased in the presence of a competitive inhibitor. Uncompetitive inhibitors generally bind to the enzyme-substrate complex or to a site that becomes available after substrate is bound at the active site and may distort the active site. Both the apparent K_{M} and the Vₘₐₓ are decreased in the presence of an uncompetitive inhibitor, and substrate concentration has little or no effect on inhibition. Mixed inhibitors are capable of binding both to free enzyme and to the enzyme-substrate complex and thus affect both substrate binding and catalytic activity. Non-competitive inhibition is a special case of mixed inhibition in which the inhibitor binds enzyme and enzyme-substrate complex with equal avidity, and inhibition is not affected by substrate concentration. Non-competitive inhibitors generally bind to enzyme at a region outside the active site. For additional details on enzyme inhibition see, for example, Voet et al. (2008) supra. For enzymes such as the lysyl oxidase-type enzymes, whose natural substrates (e.g., collagen, elastin) are normally present in vast excess in vivo (compared to the concentration of any inhibitor that can be achieved in vivo), noncompetitive inhibitors are advantageous, since inhibition is independent of substrate concentration.

Antibodies

In certain embodiments, a modulator of a lysyl oxidase-type enzyme is an antibody. In additional embodiments, an antibody is an inhibitor of the activity of a lysyl oxidase-type enzyme.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CHO of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody is one that has been identified and separated and/or recovered from a component of its natural environment. Components of its natural environment may include enzymes, hormones, and other proteinaceous or nonproteinaceous solutes. In some embodiments, an isolated antibody is purified (1) to greater than 95% by weight of antibody as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” includes an antibody in situ within recombinant cells, since at least one component of the antibody's natural environment will not be present. In certain embodiments, isolated antibody is prepared by at least one purification step.

In some embodiments, an antibody is a humanized antibody or a human antibody. Humanized antibodies include human immununoglobulins (recipient antibody) in which residues from a complementary determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies can also comprise residues that are found neither in the recipient antibody nor in the imported CDR or framework sequences. In certain embodiments, a humanized antibody comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies can also include immunoglobulin fragments, such as Fv, Fab, Fab′, F(ab′)₂ or other antigen-binding subsequences of antibodies.

The humanized antibody can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies are known in the art. Generally, a humanized antibody has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies include chimeric antibodies (U.S. Pat. No. 4,816,567), wherein substantially less than an intact human variable domain has been substituted by the corresponding sequence from a non-human species. In certain embodiments, humanized antibodies are human antibodies in which some CDR residues and optionally some framework region residues are substituted by residues from analogous sites in rodent antibodies (e.g., murine monoclonal antibodies).

Human antibodies can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

Antibodies can be affinity matured using known selection and/or mutagenesis methods as described above. In some embodiments, affinity matured antibodies have an affinity which is five times or more, ten times or more, twenty times or more, or thirty times or more than that of the starting antibody (generally murine, rabbit, chicken, humanized or human) from which the matured antibody is prepared.

An antibody can also be a bispecific antibody. Bispecific antibodies are monoclonal, and may be human or humanized antibodies that have binding specificities for at least two different antigens. In the present case, the two different binding specificities can be directed to two different lysyl oxidase-type enzymes, or to two different epitopes on a single lysyl oxidase-type enzyme.

An antibody as disclosed herein can also be an immunoconjugate. Such immunoconjugates comprise an antibody (e.g., to a lysyl oxidase-type enzyme) conjugated to a second molecule, such as a reporter An immunoconjugate can also comprise an antibody conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody that “specifically binds to” or is “specific for” a particular polypeptide or an epitope on a particular polypeptide is one that binds to that particular polypeptide or epitope without substantially binding to any other polypeptide or polypeptide epitope. In some embodiments, an antibody of the present disclosure specifically binds to its target with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM; in the form of monoclonal antibody, scFv, Fab, or other form of antibody measured at a temperature of about 4° C., 25° C., 37° C. or 42° C.

In certain embodiments, an antibody of the present disclosure binds to one or more processing sites (e.g., sites of proteolytic cleavage) in a lysyl oxidase-type enzyme, thereby effectively blocking processing of the proenzyme or preproenzyme to the catalytically active enzyme, thereby reducing the activity of the lysyl oxidase-type enzyme.

In certain embodiments, an antibody according to the present disclosure binds to human LOX with a greater binding affinity, for example, 10 times, at least 100 times, or even at least 1000 times greater, than its binding affinity to other lysyl oxidase-type enzymes, e.g., LOXL1, LOXL2, LOXL3, and LOXL4.

In additional embodiments, an antibody according to the present disclosure binds to human LOXL2 with a greater binding affinity, for example, 10 times, at least 100 times, or even at least 1000 times greater, than its binding affinity to other lysyl oxidase-type enzymes, e.g., LOX, LOXL1, LOXL3, and LOXL4.

In certain embodiments, an antibody according to the present disclosure is a non-competitive inhibitor of the catalytic activity of a lysyl oxidase-type enzyme. In certain embodiments, an antibody according to the present disclosure binds outside the catalytic domain of a lysyl oxidase-type enzyme. In certain embodiments, an antibody according to the present disclosure binds to the SRCR4 domain of LOXL2. In certain embodiments, an anti-LOXL2 antibody that binds to the SRCR4 domain of LOXL2 and functions as a non-competitive inhibitor is the AB0023 antibody, described in co-owned U.S. Patent Application Publications No. US 2009/0053224 and US 2009/0104201. In certain embodiments, an anti-LOXL2 antibody that binds to the SRCR4 domain of LOXL2 and functions as a non-competitive inhibitor is the AB0024 antibody (a human version of the AB0023 antibody), described in co-owned U.S. Patent Application Publications No. US 2009/0053224 and US 2009/0104201.

Optionally, an antibody according to the present disclosure not only binds to a lysyl oxidase-type enzyme but also reduces or inhibits uptake or internalization of the lysyl oxidase-type enzyme, e.g., via integrin beta 1 or other cellular receptors or proteins. Such an antibody could, for example, bind to extracellular matrix proteins, cellular receptors, and/or integrins.

Exemplary antibodies that recognize lysyl oxidase-type enzymes, and additional disclosure relating to antibodies to lysyl oxidase-type enzymes, is provided in co-owned U.S. Patent Application Publications No. US 2009/0053224 and US 2009/0104201, the disclosures of which are incorporated by reference for the purposes of describing antibodies to lysyl oxidase-type enzymes, their manufacture, and their use.

Polynucleotides for Modulating Expression of Lysyl Oxidase-Type Enzymes

Antisense

Modulation (e.g., inhibition) of a lysyl oxidase-type enzyme can be effected by down-regulating expression of the lysyl oxidase enzyme at either the transcriptional or translational level. One such method of modulation involves the use of antisense oligo- or polynucleotides capable of sequence-specific binding with a mRNA transcript encoding a lysyl oxidase-type enzyme.

Binding of an antisense oligonucleotide (or antisense oligonucleotide analogue) to a target mRNA molecule can lead to the enzymatic cleavage of the hybrid by intracellular RNase H. In certain cases, formation of an antisense RNA-mRNA hybrid can interfere with correct splicing. In both cases, the number of intact, functional target mRNAs, suitable for translation, is reduced or eliminated. In other cases, binding of an antisense oligonucleotide or oligonucleotide analogue to a target mRNA can prevent (e.g., by steric hindrance) ribosome binding, thereby preventing translation of the mRNA.

Antisense oligonucleotides can comprise any type of nucleotide subunit, e.g., they can be DNA, RNA, analogues such as peptide nucleic acids (PNA), or mixtures of the preceding. RNA oligonucleotides form a more stable duplex with a target mRNA molecule, but the unhybridized oligonucleotides are less stable intracellularly than other types of oligonucleotides and oligonucleotide analogues. The instability of RNA oligonucleotides can be mitigated by expressing them inside a cell using vectors designed for this purpose. This approach can be used, for example, when attempting to target a mRNA that encodes an abundant and long-lived protein.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Algorithms for identifying oligonucleotide sequences with the highest predicted binding affinity for their target mRNA, based on a thermodynamic cycle that accounts for the energy of structural alterations in both the target mRNA and the oligonucleotide, are available. For example, Walton et al. (1999) Biotechnol. Bioeng. 65:1-9 used such a method to design antisense oligonucleotides directed to rabbit β-globin (RBG) and mouse tumor necrosis factor-α (TNF α) transcripts. The same research group has also reported that the antisense activity of rationally selected oligonucleotides against three model target mRNAs (human lactate dehydrogenase A and B and rat gp130) in cell culture proved effective in almost all cases. This included tests against three different targets in two cell types using oligonucleotides made by both phosphodiester and phosphorothioate chemistries.

In addition, several approaches for designing and predicting efficiency of specific oligonucleotides using an in vitro system are available. See, e.g., Matveeva et al. (1998) Nature Biotechnology 16:1374-1375.

An antisense oligonucleotide according to the present disclosure includes a polynucleotide or a polynucleotide analogue of at least 10 nucleotides, for example, between 10 and 15, between 15 and 20, at least 17, at least 18, at least 19, at least 20, at least 22, at least 25, at least 30, or even at least 40 nucleotides. Such a polynucleotide or polynucleotide analogue is able to anneal or hybridize (i.e., form a double-stranded structure on the basis of base complementarity) in vivo, under physiological conditions, with a mRNA encoding a lysyl oxidase-type enzyme, e.g., LOX or LOXL2.

Antisense oligonucleotides according to the present disclosure can be expressed from a nucleic acid construct administered to a cell or tissue. Optionally, expression of the antisense sequences is controlled by an inducible promoter, such that expression of antisense sequences can be switched on and off in a cell or tissue. Alternatively antisense oligonucleotides can be chemically synthesized and administered directly to a cell or tissue, as part of, for example, a pharmaceutical composition.

Antisense technology has led to the generation of highly accurate antisense design algorithms and a wide variety of oligonucleotide delivery systems, thereby enabling those of ordinary skill in the art to design and implement antisense approaches suitable for downregulating expression of known sequences. For additional information relating to antisense technology, see, for example, Lichtenstein et al., “Antisense Technology: A Practical Approach,” Oxford University Press, 1998.

Small RNA and RNAi

Another method for inhibition of the activity of a lysyl oxidase-type enzyme is RNA interference (RNAi), an approach which utilizes double-stranded small interfering RNA (siRNA) molecules that are homologous to a target mRNA and lead to its degradation. Carthew (2001) Curr. Opin. Cell. Biol. 13:244-248.

RNA interference is typically a two-step process. In the first step, which is termed as the initiation step, input dsRNA is digested into 21-23 nucleotide (nt) small interfering RNAs (siRNAs), probably by the action of Dicer, a member of the RNase III family of double-strand-specific ribonucleases, which cleaves double-stranded RNA in an ATP-dependent manner. Input RNA can be delivered, e.g., directly or via a transgene or a virus. Successive cleavage events degrade the RNA to 19-21 bp duplexes (siRNA), each with 2-nucleotide 3′ overhangs. Hutvagner et al. (2002) Curr. Opin. Genet. Dev. 12:225-232; Bernstein (2001) Nature 409:363-366.

In the second, effector step, siRNA duplexes bind to a nuclease complex to form the RNA-induced silencing complex (RISC). An ATP-dependent unwinding of the siRNA duplex is required for activation of the RISC. The active RISC (containing a single siRNA and an RNase) then targets the homologous transcript by base pairing interactions and typically cleaves the mRNA into fragments of approximately 12 nucleotides, starting from the 3′ terminus of the siRNA. Hutvagner et al., supra; Hammond et al. (2001) Nat. Rev. Gen. 2:110-119; Sharp (2001) Genes. Dev. 15:485-490.

RNAi and associated methods are also described in Tuschl (2001) Chem. Biochem. 2:239-245; Cullen (2002) Nat. Immunol. 3:597-599; and Brantl (2002) Biochem. Biophys. Acta. 1575:15-25.

An exemplary strategy for synthesis of RNAi molecules suitable for use with the present disclosure, as inhibitors of the activity of a lysyl oxidase-type enzyme, is to scan the appropriate mRNA sequence downstream of the start codon for AA dinucleotide sequences. Each AA, plus the downstream (i.e., 3′ adjacent) 19 nucleotides, is recorded as a potential siRNA target site. Target sites in coding regions are preferred, since proteins that bind in untranslated regions (UTRs) of a mRNA, and/or translation initiation complexes, may interfere with binding of the siRNA endonuclease complex. Tuschl (2001) supra. It will be appreciated though, that siRNAs directed at untranslated regions can also be effective, as has been demonstrated in the case wherein siRNA directed at the 5′ UTR of the GAPDH gene mediated about 90% decrease in cellular GAPDH mRNA and completely abolished protein level (Ambion, Austin, Tex.). Once a set of potential target sites is obtained, as described above, the sequences of the potential targets are compared to an appropriate genomic database (e.g., human, mouse, rat etc.) using a sequence alignment software, (such as the BLAST software available from NCBI). Potential target sites that exhibit significant homology to other coding sequences are rejected.

Qualifying target sequences are selected as templates for siRNA synthesis. Selected sequences can include those with low G/C content as these have been shown to be more effective in mediating gene silencing, compared to those with G/C content higher than 55%. Several target sites can be selected along the length of the target gene for evaluation. For better evaluation of the selected siRNAs, a negative control is used in conjunction. Negative control siRNA can include a sequence with the same nucleotide composition as a test siRNA, but lacking significant homology to the genome. Thus, for example, a scrambled nucleotide sequence of the siRNA may be used, provided it does not display any significant homology to any other gene.

The siRNA molecules of the present disclosure can be transcribed from expression vectors which can facilitate stable expression of the siRNA transcripts once introduced into a host cell. These vectors are engineered to express small hairpin RNAs (shRNAs), which are processed in vivo into siRNA molecules capable of carrying out gene-specific silencing. See, for example, Brummelkamp et al. (2002) Science 296:550-553; Paddison et al (2002) Genes Dev. 16:948-958; Paul et al. (2002) Nature Biotech. 20:505-508; Yu et al. (2002) Proc. Natl. Acad. Sci. USA 99:6047-6052.

Small hairpin RNAs (shRNAs) are single-stranded polynucleotides that form a double-stranded, hairpin loop structure. The double-stranded region is formed from a first sequence that is hybridizable to a target sequence, such as a polynucleotide encoding a lysyl oxidase-type enzyme (e.g., a LOX or LOXL2 mRNA) and a second sequence that is complementary to the first sequence. The first and second sequences form a double stranded region; while the un-base-paired linker nucleotides that lie between the first and second sequences form a hairpin loop structure. The double-stranded region (stem) of the shRNA can comprise a restriction endonuclease recognition site.

A shRNA molecule can have optional nucleotide overhangs, such as 2-bp overhangs, for example, 3′ UU-overhangs. While there may be variation, stem length typically ranges from approximately 15 to 49, approximately 15 to 35, approximately 19 to 35, approximately 21 to 31 bp, or approximately 21 to 29 bp, and the size of the loop can range from approximately 4 to 30 bp, for example, about 4 to 23 bp.

For expression of shRNAs within cells, plasmid vectors can be employed that contain a promoter (e.g., the RNA Polymerase III H1-RNA promoter or the U6 RNA promoter), a cloning site for insertion of sequences encoding the shRNA, and a transcription termination signal (e.g., a stretch of 4-5 adenine-thymidine base pairs). Polymerase III promoters generally have well-defined transcriptional initiation and termination sites, and their transcripts lack poly(A) tails. The termination signal for these promoters is defined by the polythymidine tract, and the transcript is typically cleaved after the second encoded uridine. Cleavage at this position generates a 3′ UU overhang in the expressed shRNA, which is similar to the 3′ overhangs of synthetic siRNAs. Additional methods for expressing shRNA in mammalian cells are described in the references cited above.

An example of a suitable shRNA expression vector is pSUPER™ (Oligoengine, Inc., Seattle, Wash.), which includes the polymerase-III H1-RNA gene promoter with a well defined transcriptional startsite and a termination signal consisting of five consecutive adenine-thymidine pairs. Brummelkamp et al., supra. The transcription product is cleaved at a site following the second uridine (of the five encoded by the termination sequence), yielding a transcript which resembles the ends of synthetic siRNAs, which also contain nucleotide overhangs. Sequences to be transcribed into shRNA are cloned into such a vector such that they will generate a transcript comprising a first sequence complementary to a portion of a mRNA target (e.g., a mRNA encoding a lysyl oxidase-type enzyme), separated by a short spacer from a second sequence comprising the reverse complement of the first sequence. The resulting transcript folds back on itself to form a stem-loop structure, which mediates RNA interference (RNAi).

Another suitable siRNA expression vector encodes sense and antisense siRNA under the regulation of separate pol III promoters. Miyagishi et al. (2002) Nature Biotech. 20:497-500. The siRNA generated by this vector also includes a five thymidine (T5) termination signal.

siRNAs, shRNAs and/or vectors encoding them can be introduced into cells by a variety of methods, e.g., lipofection. Vector-mediated methods have also been developed. For example, siRNA molecules can be delivered into cells using retroviruses. Delivery of siRNA using retroviruses can provide advantages in certain situations, since retroviral delivery can be efficient, uniform and immediately selects for stable “knock-down” cells. Devroe et al. (2002) BMC Biotechnol. 2:15.

Recent scientific publications have validated the efficacy of such short double stranded RNA molecules in inhibiting target mRNA expression and thus have clearly demonstrated the therapeutic potential of such molecules. For example, RNAi has been utilized for inhibition in cells infected with hepatitis C virus (McCaffrey et al. (2002) Nature 418:38-39), HIV-1 infected cells (Jacque et al. (2002) Nature 418:435-438), cervical cancer cells (Jiang et al. (2002) Oncogene 21:6041-6048) and leukemic cells (Wilda et al. (2002) Oncogene 21:5716-5724).

Methods for Modulating Expression of Lysyl Oxidase-Type Enzymes

Another method for modulating the activity of a lysyl oxidase-type enzyme is to modulate the expression of its encoding gene, leading to lower levels of activity if gene expression is repressed, and higher levels if gene expression is activated. Modulation of gene expression in a cell can be achieved by a number of methods.

For example, oligonucleotides that bind genomic DNA (e.g., regulatory regions of a lysyl oxidase-type gene) by strand displacement or by triple-helix formation can block transcription, thereby preventing expression of a lysyl oxidase-type enzyme. In this regard, the use of so-called “switch back” chemical linking, in which an oligonucleotide recognizes a polypurine stretch on one strand of its target and a homopurine sequence on the other strand, has been described. Triple-helix formation can also be obtained using oligonucleotides containing artificial bases, thereby extending binding conditions with regard to ionic strength and pH.

Modulation of transcription of a gene encoding a lysyl oxidase-type enzyme can also be achieved, for example, by introducing into cell a fusion protein comprising a functional domain and a DNA-binding domain, or a nucleic acid encoding such a fusion protein. A functional domain can be, for example, a transcriptional activation domain or a transcriptional repression domain. Exemplary transcriptional activation domains include VP16, VP64 and the p65 subunit of NF-KB; exemplary transcriptional repression domains include KRAB, KOX and v-erbA.

In certain embodiments, the DNA-binding domain portion of such a fusion protein is a sequence-specific DNA-binding domain that binds in or near a gene encoding a lysyl oxidase-type enzyme, or in a regulatory region of such a gene. The DNA-binding domain can either naturally bind to a sequence at or near the gene or regulatory region, or can be engineered to so bind. For example, the DNA-binding domain can be obtained from a naturally-occurring protein that regulates expression of a gene encoding a lysyl oxidase-type enzyme. Alternatively, the DNA-binding domain can be engineered to bind to a sequence of choice in or near a gene encoding a lysyl oxidase-type enzyme or in a regulatory region of such a gene.

In this regard, the zinc finger DNA-binding domain is useful, inasmuch as it is possible to engineer zinc finger proteins to bind to any DNA sequence of choice. A zinc finger binding domain comprises one or more zinc finger structures. Miller et al. (1985) EMBO J 4:1609-1614; Rhodes (1993) Scientific American, February: 56-65; U.S. Pat. No. 6,453,242. Typically, a single zinc finger is about 30 amino acids in length and contains four zinc-coordinating amino acid residues. Structural studies have demonstrated that the canonical (C₂H₂) zinc finger motif contains two beta sheets (held in a beta turn which generally contains two zinc-coordinating cysteine residues) packed against an alpha helix (generally containing two zinc coordinating histidine residues).

Zinc fingers include both canonical C₂H₂ zinc fingers (i.e., those in which the zinc ion is coordinated by two cysteine and two histidine residues) and non-canonical zinc fingers such as, for example, C₃H zinc fingers (those in which the zinc ion is coordinated by three cysteine residues and one histidine residue) and C₄ zinc fingers (those in which the zinc ion is coordinated by four cysteine residues). Non-canonical zinc fingers can also include those in which an amino acid other than cysteine or histidine is substituted for one of these zinc-coordinating residues. See e.g., WO 02/057293 (Jul. 25, 2002) and US 2003/0108880 (Jun. 12, 2003).

Zinc finger binding domains can be engineered to have a novel binding specificity, compared to a naturally-occurring zinc finger protein; thereby allowing the construction of zinc finger binding domains engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. Engineering methods include, but are not limited to, rational design and various types of empirical selection methods.

Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,030,215; 7,067,617; U.S. Patent Application Publication Nos. 2002/0165356; 2004/0197892; 2007/0154989; 2007/0213269; and International Patent Application Publication Nos. WO 98/53059 and WO 2003/016496.

Exemplary selection methods, including phage display, interaction trap, hybrid selection and two-hybrid systems, are disclosed in U.S. Pat. Nos. 5,789,538; 5,925,523; 6,007,988; 6,013,453; 6,140,466; 6,200,759; 6,242,568; 6,410,248; 6,733,970; 6,790,941; 7,029,847 and 7,297,491; as well as U.S. Patent Application Publication Nos. 2007/0009948 and 2007/0009962; WO 98/37186; WO 01/60970 and GB 2,338,237.

Enhancement of binding specificity for zinc finger binding domains has been described, for example, in U.S. Pat. No. 6,794,136 (Sep. 21, 2004). Additional aspects of zinc finger engineering, with respect to inter-finger linker sequences, are disclosed in U.S. Pat. No. 6,479,626 and U.S. Patent Application Publication No. 2003/0119023. See also Moore et al. (2001a) Proc. Natl. Acad. Sci. USA 98:1432-1436; Moore et al. (2001b) Proc. Natl. Acad. Sci. USA 98:1437-1441 and WO 01/53480.

Further details on the use of fusion proteins comprising engineered zinc finger DNA-binding domains are found, for example, in U.S. Pat. Nos. 6,534,261; 6,607,882; 6,824,978; 6,933,113; 6,979,539; 7,013,219; 7,070,934; 7,163,824 and 7,220,719.

Additional methods for modulating the expression of a lysyl oxidase-type enzyme include targeted mutagenesis, either of the gene or of a regulatory region that controls expression of the gene. Exemplary methods for targeted mutagenesis using fusion proteins comprising a nuclease domain and an engineered DNA-binding domain are provided, for example, in U.S. patent application publications 2005/0064474; 2007/0134796; and 2007/0218528.

Formulations, Kits and Routes of Administration

Therapeutic compositions comprising compounds identified as modulators of the activity of a lysyl oxidase-type enzyme (e.g., inhibitors or activators of a lysyl oxidase-type enzyme) are also provided. Such compositions typically comprise the modulator and a pharmaceutically acceptable carrier. Supplementary active compounds can also be incorporated into the compositions.

As used herein, the term “therapeutically effective amount” or “effective amount” refers to an amount of a therapeutic agent that when administered alone or in combination with another therapeutic agent to a cell, tissue, or subject (e.g., a mammal such as a human or a non-human animal such as a primate, rodent, cow, horse, pig, sheep, etc.) is effective to prevent or ameliorate the disease condition or the progression of the disease. A therapeutically effective dose further refers to that amount of the compound sufficient to result in full or partial amelioration of symptoms, e.g., treatment, healing, prevention or amelioration of the relevant medical condition, or an increase in rate of treatment, healing, prevention or amelioration of such conditions. A therapeutically effective amount of, for example, an inhibitor of the activity of a lysyl oxidase-type enzyme varies with the type of disease or disorder, extensiveness of the disease or disorder, and size of the organism suffering from the disease or disorder.

The therapeutic compositions disclosed herein are useful for, inter alia, reducing fibrotic damage, inhibiting tumor growth, and inhibiting cancer metastasis. Accordingly, a “therapeutically effective amount” of a modulator (e.g., inhibitor) of the activity of a lysyl oxidase-type enzyme is an amount that results in reduction of fibrotic damage, reduction in tumor growth, and/or decrease in metastasis. For example, when the inhibitor of a lysyl oxidase enzyme is an antibody and the antibody is administered in vivo, normal dosage amounts may vary from about 10 ng/kg to up to 100 mg/kg of mammal body weight or more per day, for example, about 1 μg/kg/day to 50 mg/kg/day, optionally about 100 μg/kg/day to 20 mg/kg/day, 500 μg/kg/day to 10 mg/kg/day, or 1 mg/kg/day to 10 mg/kg/day, depending upon, e.g., body weight, route of administration, severity of disease, etc.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The disclosed therapeutic compositions further include pharmaceutically acceptable materials, compositions or vehicle, such as a liquid or solid filler, diluent, excipient, solvent or encapsulating material, i.e., carriers. These carriers are involved in transporting the subject modulator from one organ, or region of the body, to another organ, or region of the body. Each carrier should be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Another aspect of the present disclosure relates to kits for carrying out the administration of a modulator of the activity of a lysyl oxidase-type enzyme. In one embodiment, a kit comprises an inhibitor of the activity of a lysyl oxidase-type enzyme (e.g., an inhibitor of LOX or LOXL2) formulated in a pharmaceutical carrier, optionally containing one or more additional therapeutic agents, as appropriate.

The formulation and delivery methods can be adapted according to the site(s) and degree of fibrotic damage, tumor growth, or metastasis. Exemplary formulations include, but are not limited to, those suitable for parenteral administration, e.g., intravenous, intra-arterial, intra-ocular, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compounds of the disclosure will vary according to the extent and severity of the need for treatment, the activity of the administered composition, the general health of the subject, and other considerations well known to the skilled artisan.

In additional embodiments, the compositions described herein are delivered locally. Localized delivery allows for the delivery of the composition non-systemically, for example, to a wound, tumor or fibrotic area; thereby reducing the body burden of the composition, compared to systemic delivery. Accordingly, the present disclosure provides formulations and delivery methods for both systemic and local delivery (e.g, delivery to a tumor or to fibrotic tissue).

Nucleic acids encoding antibodies to a lysyl oxidase-type enzyme (or any other type of modulator; e.g., inhibitor; of the activity of a lysyl oxidase-type enzyme, e.g., a ribozyme, siRNA, shRNA or microRNA) can optionally be encapsidated in a viral vector. A number of viral vectors are known in the art, including parvoviruses, papovaviruses, adenoviruses, herpesviruses, poxviruses, retroviruses and lentiviruses.

### EXAMPLES

### Example 1

### Human Umbilical Vein Endothelial Cell (HUVEC) Vasculogenesis/Angiogenesis Assay

Summary

Studies were conducted to determine the effects of anti-LOXL2 antibody AB0023 in an in vitro vasculogenesis/angiogenesis assay. For this purpose, a dose range of AB0023 was tested alongside VEGF (an angiogenesis promoter), suramin (an angiogenesis inhibitor) and media alone in a HUVECs based in vitro system. The assay was run for 11 days with media changes on days 1, 4, 7 and 9. After 11 days, wells were fixed with 70% ethanol and vessels were tested for expression of CD31, an endothelial marker. Images of 4 random fields per well were taken and the number of branch points, number of vessels and total vessel length was calculated for each well using a specialist software analysis package.

The results of this analysis indicated that AB0023 significantly inhibited the formation of vessels in this assay, as measured by the number of vessel branch points, number of vessels, and total vessel length. The highest dose (50 μg/ml) completely abolished vessel formation, whereas 10, 5 and 1 μg/ml doses significantly reduced all three parameters relative to the control wells (p<0.05, One way ANOVA). The two lowest doses of test item (0.5 and 0.1 μg/ml) also caused a reduction in the three vessel parameters measured but these were not statistically different to the control wells. Suramin and VEGF caused a significant decrease and increase in all three vessel parameters, respectively, relative to the control wells (p<0.05, One way ANOVA).

These results lead to the conclusion that AB0023 significantly inhibits the formation of new blood vessels at the four highest doses tested (50, 10, 5 and 1 μg/ml) in this HUVEC based in vitro vasculogenesis/angiogenesis assay.

Cell Culture and Dosing

HUVEC and matrix-producing cells (a feeder layer of human fibroblasts) were cultured in 24-well plates in endothelial growth medium until the cultures demonstrated the earliest stages of tubule formation. At this point (day 1), fresh endothelial cell growth medium containing the test item and controls, as detailed in Table 1, was added to wells at 0.5 ml per well. Test item and controls were added in duplicate or triplicate according to Table 1, and media-only added in triplicate acted as baseline endothelial growth control. The plate was then cultured at 37° C. and 5% CO₂. On days 4, 7 and 9 the medium was removed from all wells and carefully replaced with 0.5 ml of fresh medium containing the test item and controls as detailed in Table 1. On day 11, the plates were fixed and labeled for CD31 as detailed below. On each day of the experiment the plate was visualized using an inverted microscope to ensure normal growth cells and no contamination.

TABLE 1

| | | Item | | | Time of |
|---|---|---|---|---|---|
| Group | N = | administered | Dose | Administration schedule | harvest |
| 1 | 3 | AB0023 | 0.1 μg/ml | Add on Day 1. Change media/test | Day 11 |
| | | | | item on Days 4, 7, and 9. | |
| 2 | 3 | AB0023 | 0.5 μg/ml | Add on Day 1. Change media/test | Day 11 |
| | | | | item on Days 4, 7, and 9. | |
| 3 | 3 | AB0023 | 1.0 μg/ml | Add on Day 1. Change media/test | Day 11 |
| | | | | item on Days 4, 7, and 9. | |
| 4 | 3 | AB0023 | 5.0 μg/ml | Add on Day 1. Change media/test | Day 11 |
| | | | | item on Days 4, 7, and 9. | |
| 5 | 3 | AB0023 | 10.0 μg/ml | Add on Day 1. Change media/test | Day 11 |
| | | | | item on Days 4, 7, and 9. | |
| 6 | 2 | AB0023 | 50.0 μg/ml | Add on Day 1. Change media/test | Day 11 |
| | | | | item on Days 4, 7, and 9. | |
| 7 | 2 | Suramin | 20 μM | Add on Day 1. Change media/test | Day 11 |
| | | | | item on Days 4, 7, and 9. | |
| 8 | 2 | VEGF | 2 ng/ml | Add on Day 1. Change media/test | Day 11 |
| | | | | item on Days 4, 7, and 9. | |
| 9 | 2 | Media only | Not | Add on Day 1. Change media/test | Day 11 |
| | | treated | applicable | item on Days 4, 7, and 9. | |

CD31 Labeling of Vessels

After 11 days, plates were fixed by carefully aspirating medium from wells, washed with 1 ml PBS per well, and then 1 ml ice cold 70% ethanol was added for 30 minutes at room temperature. Fixative was then carefully removed and the wells were incubated with 0.5 ml mouse anti-human CD31 antibody diluted 1:400 in PBS containing 1% BSA and incubated for 60 minutes at 37° C. Wells were then washed three times with 1 ml PBS followed by incubation with 0.5 ml goat anti-mouse IgG alkaline phosphatase conjugated secondary antibody diluted 1:500 in PBS containing 1% BSA for 60 minutes at 37° C. Wells were washed a further three times with 1 ml PBS prior to incubation with 0.5 ml freshly prepared and filtered BCIP/NBT substrate for 10 minutes at 37° C. Wells were then carefully washed three times with 1 ml distilled water and left to air dry overnight.

Data Analysis

Digital images of each well were obtained using a Nikon Coolpix camera on a Leica inverted microscope at 10× magnification. Four random fields per well were imaged, producing 96 images in total. Images were then converted to BMP files and imported into an image analysis package to measure the number of vessel branches, number of vessels and total vessel length. This is performed by thresholding the image so that only CD31-stained vessels were detected, which the software then skeletonized to produce single pixel width vessels. From these, individual vessel length, total vessel length and number of vessel branch points were determined. The data were then exported to Excel to calculate mean and standard deviation for each data set. Basic statistics were carried out using a one-way ANOVA.

Results

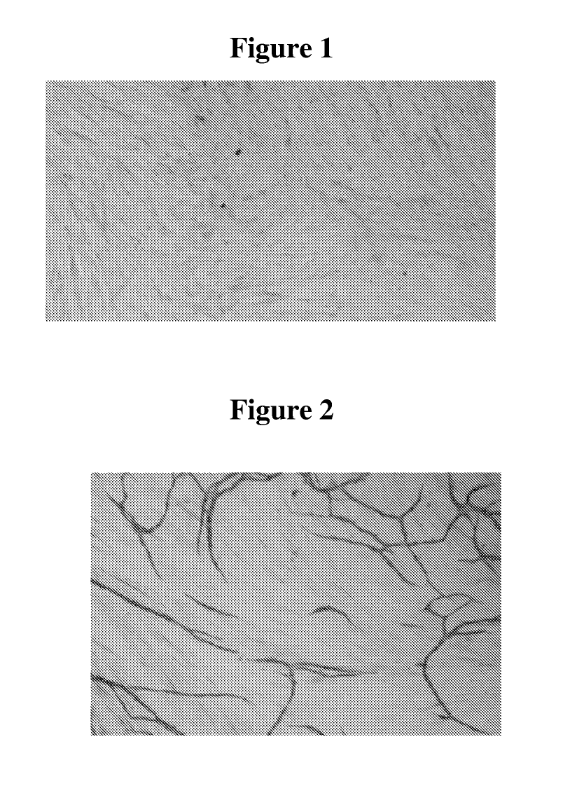

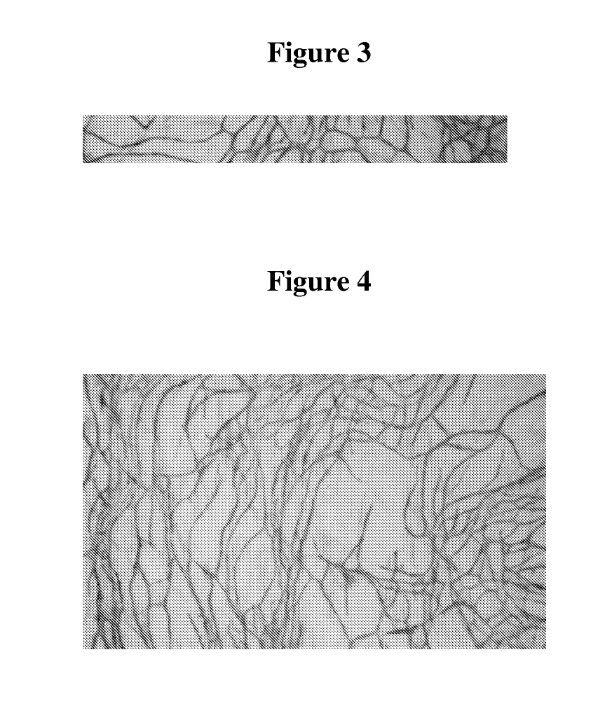

As shown in FIG. 1, the 50 μg/ml dose of AB0023 completely abolished vessel growth and formation. Due to the lack of vessels, no data on vessel number, length or branching was available for this group; hence it is omitted from the analyses discussed below. Blood vessel growth and formation was observed at all other concentrations of AB0023, but was inhibited in a dose dependent manner by the anti-LOXL2 antibody. Exposure to suramin resulted in a decrease in vessel formation (FIG. 2); while exposure to VEGF resulted in an increase in vessel formation (FIG. 4), relative to control wells (FIG. 3).

The 96 images taken of the plate were quantified using image analysis software and the data were collated. The mean and standard deviation values were then obtained for the three different parameters measured (number of vessel branch points, number of vessels and total vessel length). These results indicated that AB0023 induced a dose-dependent reduction in the number of branch points, total number of vessels, and total vessel length. Treatment with 10, 5 and 1 μg/ml of test item or 20 μM suramin caused a significant reduction in the number of branches whereas 2 ng/ml VEGF caused a significant increase in branching relative to the control group (p<0.05 one way ANOVA).

What is claimed is:

1. A method for identifying an inhibitor of LOXL2 activity, the method comprising:
(a) providing a co-culture of endothelial cells growing in the presence of stromal cells;
(b) adding a test molecule to the co-culture; and
(c) assaying the co-culture for angiogenesis or vasculogenesis;
wherein a test molecule that reduces the degree of angiogenesis or vasculogenesis in the co-culture, compared to a co-culture in the absence of the test molecule, is identified as an inhibitor of LOXL2 activity.

2. The method of claim 1, wherein the endothelial cells are human endothelial cells.

3. The method of claim 2, wherein the endothelial cells are human umbilical vein endothelial cells (HUVEC).

4. The method of claim 1, wherein the stromal cells are human cells.

5. The method of claim 1, wherein the stromal cells are fibroblasts.

6. The method of claim 1, wherein the test molecule is a polypeptide.

7. The method of claim 6, wherein the polypeptide is an antibody.

8. The method of claim 7, wherein the antibody is an anti-LOXL2 antibody.

9. The method of claim 1, wherein the test molecule is a nucleic acid.

10. The method of claim 9, wherein the nucleic acid is a siRNA.

11. The method of claim 1, wherein the test molecule is a small organic molecule with a molecular weight less than 1,000 D.

12. The method of claim 1, wherein reduction in the degree of angiogenesis or vasculogenesis is indicated by a reduction in vessel number or density.

13. The method of claim 1, wherein reduction in the degree of angiogenesis or vasculogenesis is indicated by a reduction in vessel length.

14. The method of claim 1, wherein reduction in the degree of angiogenesis or vasculogenesis is indicated by a reduction in the degree of vessel branching.

15. The method of claim 1, wherein reduction in the degree of angiogenesis or vasculogenesis is indicated by a reduction in levels of CD31.
